(19) United States
(12) Patent Application Publication
(10) Pub. No.: US 2023/0320839 A1
(43) Pub. Date: Oct. 12, 2023

(54) DEVICE FOR INTERFACING FILAMENTOUS OR FIBROUS STRUCTURES WITH A REAL OR SIMULATED BIOLOGICAL TISSUE

(71) Applicant: Alma Mater Studiorum - Università di Bologna, Bologna (IT)

(72) Inventors: Alberto SENSINI, Bologna (IT); Andrea ZUCCHELLI, Bologna (IT); Luca CRISTOFOLINI, Bologna (IT); Chira GUALANDI, Bologna (IT); Maria Letizia FOCARETE, Bologna (IT)

(73) Assignee: Alma Mater Studiorum - Università di Bologna, Bologna (IT)

(21) Appl. No.: 18/025,138

(22) PCT Filed: Sep. 8, 2021
(86) PCT No.: PCT/IB2021/058153

§ 371 Date: Mar. 7, 2023

(30) Foreign Application Priority Data
Sep. 9, 2020 (IT) 102020000021313

(51) Int. Cl. A61F 2/08

(52) U.S. Cl. CPC A61F 2/0811; A61F 2002/0086

(57) ABSTRACT

A device for interfacing at least one filamentous structure, with real or simulated biological tissue and a system for regeneration, repair, replacement, or simulation of tendon and/or ligamentous tissue. The device comprises one or more bodies for anchoring a filamentous structure. The one or more bodies may include at least one capstan for wrapping the filamentous structure, and at least one porous portion having a trabecular structure. The system comprises the device and at least one filamentous structure having a plurality of nanofiber assemblies that are obtained by electrospinning. The plurality of assemblies may be arranged to form a single bundle, with the bundle being wrapped to the capstan.

### TECHNICAL FIELD OF THE INVENTION

The present invention relates to a device for interfacing filamentous, or fibrous, structures with a real or simulated biological tissue. For the purposes of the present description, the term “filamentous or fibrous structures” means any structure comprising one or more filaments or fibers. Preferably, such filamentous structures, are hierarchical electrospun supports for regeneration, or repair, or replacement, of tendon/ligamentous tissue that, by means of the device of the present invention, can be interfaced with a real biological tissue, such as bone tissue. Therefore, the present invention further relates to a system for regeneration, or repair, or replacement, of tendon/ligamentous tissue comprising such electrospun hierarchical supports anchored to the device for interfacing.

Such filamentous structures, may, in addition, be electrospun hierarchical supports for the simulation of tendon/ligamentous and/or muscle tissue which, thanks to the device of the present invention, are interfaced, with a simulated biological tissue, in order to obtain parts of robotic systems for the simulation of the mechanical behavior of muscles such as, for example, actuators of a prosthesis. Thus, the present invention also relates to a system for simulating tendon/ligamentous and/or muscle tissue comprising such electrospun hierarchical supports anchored to the device for interfacing, as well as to a prosthesis actuator or robotic system comprising such a system.

### State of the Art

At the present state of the art, in the field of tissue engineering, supports are known for cell adhesion, proliferation and migration, whose morphology is fundamental for the final shape and structure of the tissues and organs to be reconstructed or replaced. Such supports, are also commonly known as “scaffolds”. In particular, in the field of ligament or tendon reconstruction, “scaffolds” consisting of bundles formed by nanofibers obtained by electrospinning are known. Such bundles are arranged in groups joined together, then, so as to form a single bundle and are, usually, covered by a porous membrane that keeps them aligned and compacts them (WO2018229615 A1). Such a membrane is then, in turn, formed of nanofibers. The “scaffolds” for the reconstruction of tendons and ligaments are therefore filamentous or fibrous structures that need to be anchored in vivo to the bone tissue on which the tendon/ligament to be reconstructed must be grafted. Similarly, there is also the need to anchor these filamentous or fibrous “scaffolds” to simulated biological tissue, such as, for example, that used in parts of robotic systems for the simulation of the mechanical behavior of muscles (e.g. actuators of prostheses) or of the interface between muscle tendon and bone.

In the field of tendon and ligament reconstruction, devices are currently known comprising, essentially, two elements: a screw intended to be grafted into bone tissue and an anchoring element of any artificial tendon or ligament configured to be embedded in the screw (US 2013/090731 A1). Such devices of the known art may be suitable for biological grafts or prostheses with artificial materials, but are not suitable for anchoring “scaffolds” for cellular regeneration. The latter, in fact, are supports on which, the cells that are deposited, must multiply and differentiate, depending on the tissue that is intended to regenerate, repair, or simulate. In the specific case, the cells deposited on the same “scaffold” must differentiate into fibroblasts (e.g. cells of connective tissue that makes up the tendons and ligaments) and osteoblasts (e.g. cells of bone tissue). To achieve such differentiation, with a single “scaffold”, it is necessary to reproduce, simultaneously, different mechanical conditions, or rather, different degrees of strain. These, in turn, produce different degrees of mineralization and, therefore, cells of different types. In fact, in nature, in the transition between tendon and bone, there is a gradient of deformability that is at the origin of the different degrees of mineralization of the cells, which differentiate into fibroblasts of the tendon where they are subjected to the maximum strain, into fibroblasts of the thin layer of mineralized fibrocartilage that interfaces between tendon and bone, up to osteoblasts of the bone tissue where they are subjected to the minimum strain. The devices known to the state of the art mentioned above are not able to ensure any gradient of deformability and, therefore, even if they are attached to “scaffolds” for tendon regeneration, they are not able to obtain the cell differentiation described above.

### OBJECTS AND SUMMARY OF THE INVENTION

The object of the present invention is, therefore, to provide a device for interfacing filamentous, or fibrous, structures with a real or simulated biological tissue that mimics the mechanical characteristics of the tendon/ligament/bone interface.

More particularly, the object of the present invention is to provide a device for interfacing supports or “scaffolds” for the regeneration, repair, of tendons or ligaments that, once implanted ensures a gradient of deformability such that the cells, deposited on said “scaffold”, will differentiate into the cell types characteristic of: (1) tendon/ligament tissue; (2) fibrocartilage tissue at the tendon/bone interface; and (3) bone tissue, thereby achieving complete integration of the tendon-muscle and/or ligament system to the bone.

This object is achieved by designing the device of the present invention such that it has porous zones having a trabecular structure.

For the purposes of the present invention, the term “trabecular structure” means a structure that mimics the trabecular structure of spongy bone. As is known, the latter is, in fact, a tissue consisting of thin columnar structures or trabeculae, variously oriented and intertwined with each other to delimit numerous intercommunicating cavities called areoles or medullary cavities containing bone marrow, blood vessels and nerves.

To achieve the aforementioned object, the present invention provides a device for interfacing at least one filamentous structure, with real or simulated biological tissue, comprising at least one body for anchoring the filamentous structure having: at least one capstan configured to wrap around the filamentous structure and at least one porous portion having a trabecular structure. The latter allows to mimic the structure of the bone tissue in order to better simulate the mechanical characteristics of the tendon/ligament/bone interface.

This interface is even better simulated by providing that the body for anchoring the filamentous structure comprises, not a single porous zone with homogeneous porosity, but, at least a first porous zone and a second porous zone, having different degrees of porosity.

For the purposes of the present invention, the term porosity means the percentage ratio of the total volume of pores (or voids) to the total volume of the body or material considered.

Alternatively, or in addition, to the fact that the body for anchoring the filamentous structure may comprise at least two zones of different porosity, the device of the present invention may also comprise a second body intended to interface with the bone and not having any capstan, the second body housing the body for anchoring the filamentous structure mentioned above.

More particularly, the body for anchoring the filamentous structure may be formed in the guise of tweezer with two flat, porous arms joined to each other by means of the capstan. In such a case, the second body consists of a screw with a threaded surface. As will be explained in more detail below, the tweezer are interlocked inside the screw. The latter may also be provided with two or more porous zones having different porosity with respect to each other. Therefore, if the device of the present invention is implemented in the tweezer-screw assembly, four different implementations are provided. In the first, both the tweezer and the screw are each characterized by a homogeneous porosity, but have different porosity with respect to each other. In the second case, instead, while the screw has homogeneous porosity, the tweezer has areas with different porosity. The third possibility is that both the tweezer and the screw, in addition to differing in porosity, each have zones with different porosity. The fourth possibility, finally, is that only the screw has areas with different porosity, while the tweezer do not (i.e. the two porous portions corresponding to the arms of the tweezer are characterized by having homogeneous porosity within the same arm and between different arms). In the four cases, the mechanical behavior of the screw and tweezer will be different. Since the tweezer and the screw differ, between them, both from the point of view of shape and porosity, they will be subjected to different strains, so as to simulate that gradient that, in nature, is created in the passage between tendon and bone, or more specifically in that layer of cartilage that is interposed between the tendon or ligament and the bone.

The tweezer-screw assembly may be, in particular, intended, preferably but not exclusively, for regeneration, or repair, or replacement, as well as simulation of a tendon or cylindrical ligament. In such a case, the implantation of the interface device, or rather of the system constituted by the device and the filamentous structure itself, may take place in the following way: the health care operator (e.g. surgeon) implants the screw in the bone and then inserts interlockingly, inside the screw, the tweezer, around whose capstan is wound the filamentous structure which, in this case will be, a tendon and/or ligament scaffold constituted by a bundle of nanofibers obtained by electrospinning.

A second object of the present invention is, therefore, also to provide a device for interfacing filamentous, or fibrous structures with a real biological tissue that allows a certain ease and safety in the implantation operations of the filamentous structure into the bone tissue in vivo. If the device were in fact made of a single element such as, for example, a screw, the surgeon would have to first fix the filamentous structure inside the screw and then screw the latter to the patient's bone. In this way, in addition to the fact of having to perform an uncomfortable procedure, there would be the risk of damaging the filamentous structure itself, compromising its operation. On the contrary, with the present invention, since the object being implanted consists only of the screw without any filamentous structure inside it, the operation of fixing the screw in the bone does not compromise, in any way, the integrity of the filamentous structure. The operation of interlocking the tweezer with the filamentous structure wrapped around its capstan is, in fact, less risky for the integrity of the filamentous structure than the operation of inserting the screw into the bone.

Alternatively, to the assembly comprising the tweezer and the screw, the body for anchoring the filamentous structure and, therefore, the device itself forming the subject of the present invention may comprise a plate having a plurality of capstans configured for wrapping the filamentous structure.

Said plate may be, in particular, preferably but not exclusively intended for regeneration, or repair, or replacement, as well as simulation of tendons and/or flat ligaments such as, for example, those of the rotator cuff of the scapulo-humeral joint. This plate can also be used for the regeneration, repair, replacement of spinal ligaments. Also in this case, the plate can comprise areas of different porosity in order to generate gradients of deformability and, therefore, allow the differentiation of cells into different cell types, depending on the imposed strain. More particularly, in the plate of the present invention, it is preferably envisaged that the zone farthest from the capstans, namely the zone farthest from the filamentous structure simulating the tendon(s)/link(s), has a higher porosity (e.g., larger pore size), than the zone in the proximity of the capstans (e.g., smaller pore size).

It is further disclosed herein that the present invention relates not only to a device for interfacing a filamentous structure, with real or simulated biological tissue, but also to a system for regenerating, or repairing, or replacing, or simulating tendon and/or ligament tissue comprising the above-described device and at least one filamentous structure. The latter may comprise a plurality of groups of nanofibers obtained by electrospinning, said plurality of groups being arranged to form a single bundle wrapped to the capstan (in the case of the tweezer), or wrapped to each of the capstans (in the case of the plate).

In this context, a third object of the present invention is, to, provide a system for regenerating, or repairing, or replacing, or simulating tendon and/or ligamentous tissue that promotes cell passage between nanofibers and bone without creating discontinuities in such passage. To this end, the system of the present invention may comprise nanofibers, or in general electrospun elements, within the porosity of the device, whether consisting of the tweezer alone, the screw-tweezer assembly, or the plate. In this way, no discontinuities are created between the various elements of the device (e.g., tweezer-screw assembly) and the tendon-cortical bone interface is better simulated.

These and further objects of the present invention will be made clearer by reading the following detailed description of some preferred embodiments to be understood purely as a not limiting example of the more general concepts claimed.

### BRIEF DESCRIPTION OF DRAWINGS

The following description refers to the attached drawings, wherein:

FIG. 1 is a perspective view of the first embodiment of the device of the present invention;

FIG. 2a is a front view of the first embodiment of the device of the present invention;

FIG. 2b is a side view of the first embodiment of the device of the present invention;

FIG. 3a is a first perspective view of a detail comprising the second body of the second embodiment of the device of the present invention;

FIG. 3b is a second perspective view of a detail constituted by the second body of the second embodiment of the device of the present invention;

FIG. 4 is a longitudinal section of a particular constituted by the second body of the second embodiment of the device of the present invention;

FIG. 5a is a first longitudinal section of the second embodiment of the device of the present invention;

FIG. 5b is a second longitudinal section of the second embodiment of the device of the present invention, said second longitudinal section being in a plane orthogonal to that of the first longitudinal section;

FIG. 6 is a perspective view of the fourth embodiment of the device of the present invention;

FIG. 7a is a front view of the fourth embodiment of the device of the present invention;

FIG. 7b is a front rear view of the fourth embodiment of the device of the present invention;

FIG. 8 is an exemplary schematic of a Voronoi trabecular tessellation followed by the porous portions of the device of the present invention;

FIG. 9a is a side section of a normal stress diagram obtained with a finite element model of the first embodiment of the system of the present invention;

FIG. 9b is a front section of a diagram of the equivalent Von Mises stresses obtained with a finite element model of a part of the first embodiment of the system of the present invention, said part being constituted by the body for anchoring the filament structure;

FIG. 10a is side section of a strain diagram obtained with a finite element model of the first embodiment of the system of the present invention; and

FIG. 10b is a side section of a strain diagram obtained with a finite element model of a part of the first embodiment of the system of the present invention, said part being constituted by the body for anchoring the filamentous structure and a bundle of nanofibers anchored to said body.

### DETAILED DESCRIPTION OF THE INVENTION

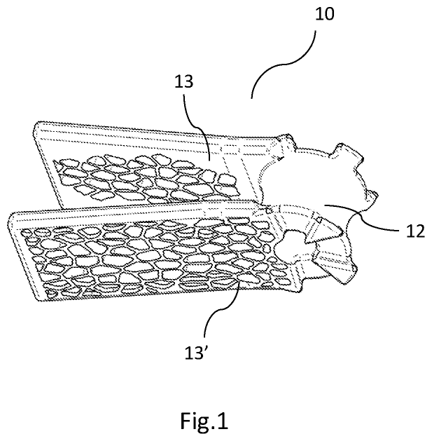

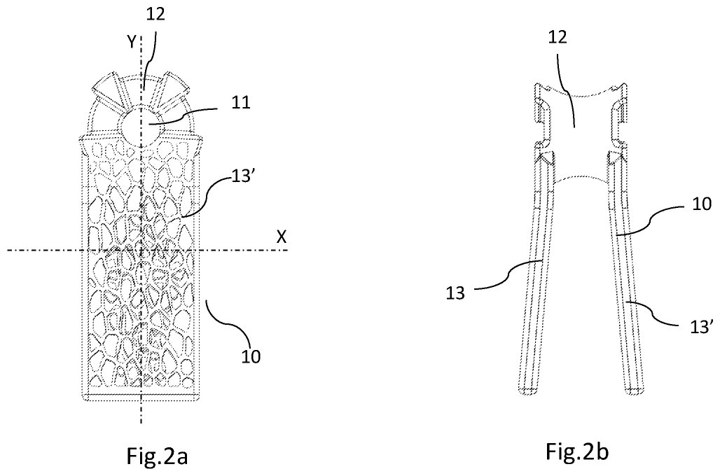

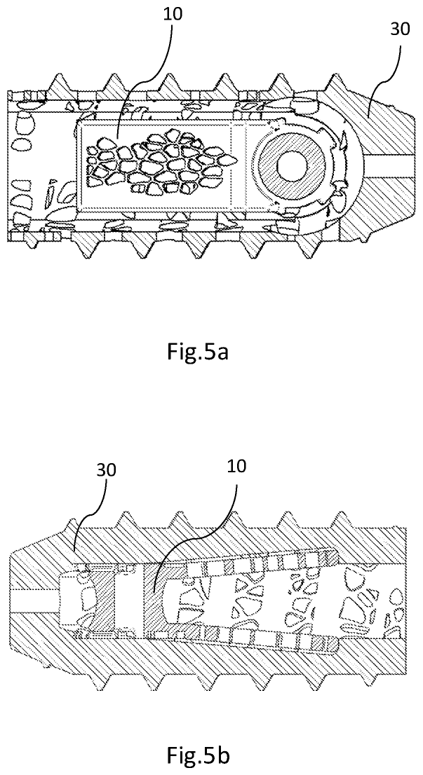

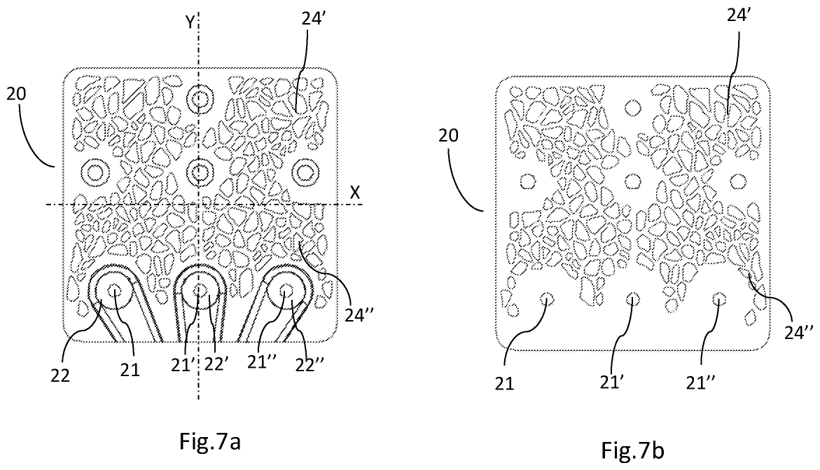

Referring to FIGS. 1, 2a, 2b, 5b, 6, 7a, 7b, the device for interfacing at least one filamentous structure, with real or simulated biological tissue, of the present invention, comprises at least one body (10, 20) for anchoring the filamentous structure, said body (10, 20) comprising:
at least one capstan (12, 22, 22′, 22′) configured for wrapping the filamentous structure;
at least one portion (13, 13′, 24) porous having a trabecular structure (300).

In the center of the at least one capstan (12, 22, 22′, 22″) there may be a hole (11, 21, 21′, 21″).

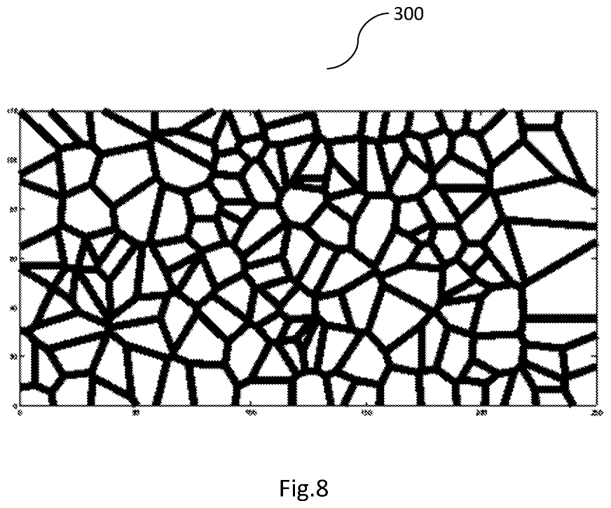

Referring to FIGS. 1, 2a, 2b, and 8, in a first embodiment of the device of the present invention, the body (10) for the anchoring the filamentous structure is conformed as a tweezer (13,13′, 12,11) comprising a first flat arm (13) and a second flat arm (13′), said arms (13, 13′) being joined to each other by means of the capstan (12).

The porous portion (13, 13′), moreover, may comprise, at least a first porous zone and a second porous zone. The difference between the porosity of the first porous zone and the porosity of the second porous zone being such as to ensure a gradient of deformability. More specifically, the first porous zone, configured for the cartilage/tendon/ligament interface, has a porosity comprised between 1% and 98% and a pore size comprised between 0.1 μm and 800 μm, and a second porous zone, configured for the bone interface, has a pore size comprised between 1 μm and 980 μm, the difference between the first porous zone and the second porous zone being comprised between 1% and 98%. However, the absolute porosity value and the pore size can vary according to the anatomical district wherein the device is directed to be implanted and according to the clinical characteristic of the patients (e.g. it is well known that the age can affect also greatly the porosity of the bone). The target application (i.e biological tissue or simulated tissue) can also influence the absolute porosity and the pore size values. In a preferred embodiment, the first porous zone (13,13′) has a porosity comprised between 2% and 95% and a pore size of 0.2 μm and 750 μm. In such preferred embodiment the pore size of the second zone is 5 μm and 950 μm. In another preferred embodiment, the first porous zone (13,13′) has a porosity comprised between 2% and 90% and a pore size of 0.5 μm and 700 μm. In such preferred embodiment the pore size of the second zone is 10 μm and 900 μm. The difference of porosity can be along the longitudinal axis (Y) of the arms (13,13′) and/or along a transversal direction (X), orthogonal to the longitudinal axis (Y) of the arms (13, 13′). The structure of the porous portion (13, 13′) follows a trabecular Voronoi tessellation (300) projected onto the surface of the arms (13, 13′) of the tweezer (10). The latter can be made of a bioresorbable and/or inert material, in case the device is used as a device for interfacing a filamentous structure with a real biological tissue (e.g., in vivo bone tissue), or made of an inert and/or conductive material, in case the device is used as a device for interfacing a filamentous structure with a simulated biological tissue (e.g., simulated tissue of a prosthesis actuator).

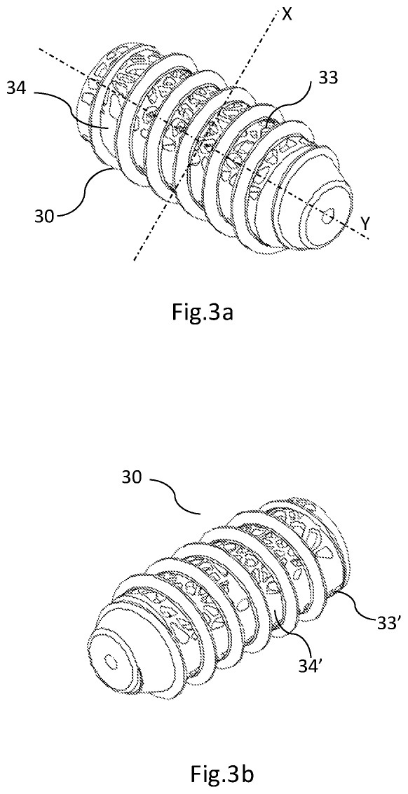

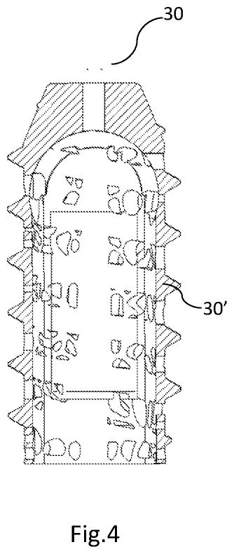

With reference to FIGS. 1, 2a, 2b, 3a, 3b, 4, 5a, 5b and 8, in a second embodiment thereof, the device of the present invention, comprises also a second hollow body (30) comprising at least one porous portion (33) having a trabecular structure (300), the body (10) for anchoring the filamentous structure being housed, and in particular embedded, within (30′) the second body (30). The trabecular structure of the second body (30) also follows a Voronoi trabecular tessellation (300) projected onto the surface of the body (30). The second body (30) is shaped like a threaded screw and has two porous portions (33, 33′) and two nonporous portions (34, 34′). Both porous portions (33,33′) of the screw (30) may also, like the porous portions (13,13′) of the tweezer (10), comprise a first porous zone for the cartilage/tendon/ligament interface and a second porous zone, for the bone interface, the difference between the porosity of the first porous zone and the porosity of the second porous zone being such that an adequate gradient of deformability is generated. More specifically, the first porous zone has a porosity comprised between 1% and 98%, and a pore size comprised between 0.1 μm and 800 μm, and the second porous zone has a pore size comprised between 1 μm and 980 μm, the difference between the first porous zone and the second porous zone being comprised between 1% and 98%. The absolute porosity value and the pore size can vary according to the anatomical district wherein the device is directed to be implanted and according to the clinical characteristic of the patients (e.g. it is well known that the age can affect also greatly the porosity of the bone). The target application (i.e biological tissue or simulated tissue) can also influence the absolute porosity and the pore size values. In a preferred embodiment, the first porous zone has a porosity comprised between 2% and 95% and a pore size of 0.2 μm and 750 μm. In such preferred embodiment the pore size of the second zone is 5 μm and 950 μm. In another preferred embodiment, the first porous zone has a porosity comprised between 2% and 90% and a pore size of 0.5 μm and 700 μm. In such preferred embodiment the pore size of the second zone is 10 μm and 900 μm. The difference of porosity can be along the longitudinal axis (Y) of the screw (30) and/or along a transversal direction (X), orthogonal to the longitudinal axis (Y) of the screw (30). Finally, the porosity of the porous portions (33, 33′) of the screw (30) may be less in the area of the screw (30) in the proximity of the capstan (12) of the tweezer (10) than the porosity in the area of the screw (30) farthest from the capstan (12) and, that is, in the proximity of the tip of the screw (30) itself.

The screw (30) may also be made of a bioresorbable and/or inert material, or made of an inert and/or conductive material depending on the application, as already described above with respect to the first embodiment of the present device. Such materials may be in particular: polyesters, polyurethanes, polyanhydrides, polycarbonates, polyamides, polyolefins and fluorinated polymers and copolymers thereof, materials of natural origin, for example polysaccharides, proteins, polyesters, polypeptides, and copolymers thereof, and/or mixtures of these materials and/or metallic materials and/or ceramic materials or combinations thereof. Moreover, the material of the screw and/or of the deformable inner element may advantageously be loaded and/or functionalized with organic and/or inorganic components capable of performing a biological function and/or modifying the physical-chemical and/or mechanical properties of the screw and/or of the deformable inner element.

In a third embodiment of the device of the present invention the body (10) for anchoring the filamentous structure comprises the body (10) for the anchoring the filamentous structure is conformed as a tweezer (13, 13′, 12, 11) comprising a first flat arm (13) and a second flat arm (13′), said arms (13, 13′) being joined to each other by means of the capstan (12).

The device comprises also a second hollow body (30) comprising at least one porous portion (33) having a trabecular structure (300), the body (10) for anchoring the filamentous structure being housed, and in particular embedded, within (30′) the second body (30). The trabecular structure of the second body (30) also follows a Voronoi trabecular tessellation (300) projected onto the surface of the body (30). The second body (30) is shaped like a threaded screw and has two porous portions (33,33′) and two nonporous portions (34, 34′). Both the tweezer (10) and the screw (30) are each characterized by a homogeneous porosity, but have different porosity with respect to each other. In other words, the porous portions (33, 33′) of the screw (30) have a porosity that differs with the porosity of the tweezer (10) such that an appropriate gradient of deformability is generated. Specifically, the porosity of the first body (10), configured for the cartilage/tendon/ligament interface, can be comprised between 1% and 98% and the pore size between 0.1 μm and 800 μm. Preferably, the porosity of the first body can vary between 2% and 95% and the pore size between 0.2 μm and 750 μm. More preferably, the porosity of the first body can vary between 2% and 90% and the pore size between 0.5 μm and 700 μm. The porosity of the second body, configured for the bone interface, can vary between 1% and 98% with a pore size comprised in the range between 1 μm and 980 μm. Preferably, the porosity of the second body can vary between 2% and 95% and the pore size between 5 μm and 950 μm. More preferably, the porosity of the second body can be comprised between 2% and 90% and the pore size between 10 μm and 900 μm. The difference between the porosity of the first body (10) and the porosity of the second body (30) can vary between 1% and 98%. More preferably, the difference between the porosity of the first body (10) and the porosity of the second body (30) is comprised in the range between 2% and 90%.

In a fourth embodiment of the device of the present invention the body (10) for the anchoring the filamentous structure is conformed as a tweezer (13, 13′, 12, 11) comprising a first flat arm (13) and a second flat arm (13′), said arms (13, 13′) being joined to each other by means of the capstan (12).

The device comprises also a second hollow body (30) comprising at least one porous portion (33) having a trabecular structure (300), the body (10) for anchoring the filamentous structure being housed, and in particular embedded, within (30′) the second body (30). The trabecular structure of the second body (30) also follows a Voronoi trabecular tessellation (300) projected onto the surface of the body (30). The second body (30) is shaped like a threaded screw and has two porous portions (33,33′) and two nonporous portions (34, 34′). The tweezer (10) has a homogeneous porosity, that is comprised between 1% and 98% and a pore size comprised between 0.1 μm and 800 μm. Preferably, the porosity of the tweezer (10) is comprised between 2% and 95% and the pore size between 0.2 μm and 750 μm. More preferably, the porosity of the tweezer (10) can vary between 2% and 90% and the pore size between 0.5 μm and 700 μm. The porous portions (33, 33′) of the screw (30) comprise a first porous zone and a second porous zone, the difference between the porosity of the first porous zone and the porosity of the second porous zone being comprised between 1 and 98%. More specifically, the first porous zone of the screw has a porosity comprised between 1% and 98%, and a pore size comprised between 0.1 μm and 800 μm. The second porous zone has a pore size comprised between 1 μm and 980 μm, the difference between the first porous zone and the second porous zone being comprised between 1% and 98%. In a preferred embodiment, the first porous zone has a porosity comprised between 2% and 95% and a pore size of 0.2 μm and 750 μm. In such preferred embodiment the pore size of the second zone is 5 μm and 950 μm. In another preferred embodiment, the first porous zone has a porosity comprised between 2% and 90% and a pore size of 0.5 μm and 700 μm. In such preferred embodiment the pore size of the second zone is 10 μm and 900 μm. The difference of porosity can be along the longitudinal axis (Y) of the screw (30) and/or along a transversal direction (X), orthogonal to the longitudinal axis (Y) of the screw (30).

In a fifth embodiment of the device of the present invention the body (10) for the anchoring the filamentous structure is conformed as a tweezer (13, 13′, 12, 11) comprising a first flat arm (13) and a second flat arm (13′), said arms (13, 13′) being joined to each other by means of the capstan (12).

The device comprises also a second hollow body (30) comprising at least one porous portion (33) having a trabecular structure (300), the body (10) for anchoring the filamentous structure being housed, and in particular embedded, within (30′) the second body (30). The trabecular structure of the second body (30) also follows a Voronoi trabecular tessellation (300) projected onto the surface of the body (30). The second body (30) is shaped like a threaded screw and has two porous portions (33,33′) and two nonporous portions (34,34′). The screw (30) has a homogeneous porosity, that is comprised between 1% and 98% and a pore size comprised between 1 μm and 980 μm. Preferably, the porosity of the screw (30) is comprised between 2% and 95% and the pore size between 5 μm and 950 μm. More preferably, the screw (30) has a porosity varying between 2% and 90% and a pore size varying between 10 μm and 900 μm. The porous portions (13, 13′) of the tweezer (10) comprise each a first porous zone and a second porous zone, the difference between the porosity of the first porous zone and the porosity of the second porous zone. More specifically, the first porous zone has a porosity comprised between 1% and 98% and a pore size comprised between 0.1 μm and 800 μm. The second porous zone has a pore size comprised between 1 μm and 980 μm, the difference between the first porous zone and the second porous zone being comprised between 1% and 98%. In a preferred embodiment, the first porous zone has a porosity comprised between 2% and 95% and a pore size of 0.2 μm and 750 μm. In such preferred embodiment the pore size of the second zone is 5 μm and 950 μm. In another preferred embodiment, the first porous zone has a porosity comprised between 2% and 90% and a pore size of 0.5 μm and 700 μm. In such preferred embodiment the pore size of the second zone is 10 μm and 900 μm.

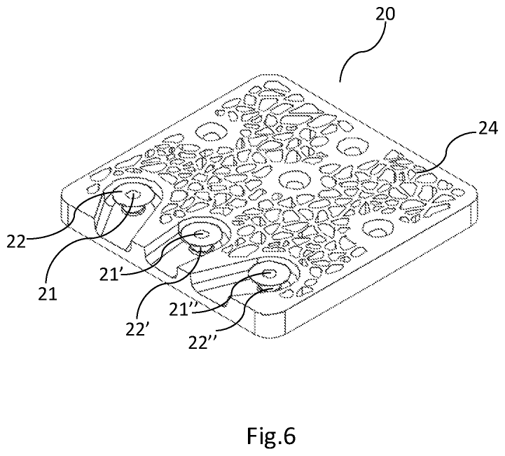

The absolute porosity value and the pore size can vary according to the anatomical district wherein the device is directed to be implanted and according to the clinical characteristic of the patients (e.g. it is well known that the age can affect also greatly the porosity of the bone). The difference of porosity can be along the longitudinal axis (Y) of the arms (13, 13′) and/or along a transversal direction (X), orthogonal to the longitudinal axis (Y) of the arms (13, 13′). With reference to FIGS. 6, 7a, 7b and 8, in a sixth embodiment thereof, the device of the present invention, has the form of a plate provided with a plurality of capstans (22, 22′, 22″). At the center of each capstan (22, 22′, 22″) there may be a hole (21, 21′, 21″). The porous portion (24) has a structure that follows a trabecular Voronoi tessellation (300) and comprises at least a first porous zone (24″) and a second porous zone (24′). The second porous zone (24′), which is located away from the capstan (22, 22′, 22″) has a higher porosity than the first porous zone (24″), which is located in the proximity of the capstan (22, 22′, 22″), and the difference between the porosity of the first porous zone (24″) and the porosity of the second porous zone (24′) is such that a gradient of deformability is provided.

More specifically, the first porous zone (24″), configured for the cartilage/tendon/ligament interface, has a porosity comprised between 1% and 98%, and a pore size comprised between 0.1 μm and 800 μm, and a second porous zone (24′), configured for the bone interface, has a pore size comprised between 1 μm and 980 μm, the difference between the first porous zone and the second porous zone being comprised between 1% and 98%. More preferably, the first porous zone has a porosity comprised between 2% and 95%, and a pore size comprised between 0.2 μm and 750 μm, and the second porous zone has a pore size comprised between 5 μm and 950 μm, the difference between the first porous zone and the second porous zone being comprised between 1% and 98%. In a preferred embodiment, the first porous zone has a porosity comprised between 2% and 90% and a pore size of 0.5 μm and 700 μm. In such preferred embodiment the pore size of the second zone is 10 μm and 900 μm. The absolute porosity value and the pore size can vary according to the anatomical district wherein the device is directed to be implanted and according to the clinical characteristic of the patients (e.g. it is well known that the age can affect also greatly the porosity of the bone). The target application (i.e biological tissue or simulated tissue) can also influence the absolute porosity and the pore size values. The difference of porosity can be along the longitudinal axis (Y) of the plate (20) and/or along a transversal direction (X), orthogonal to the longitudinal axis (Y) of the plate (20).

The plate (20) may also be made of a bioresorbable and/or inert material or made of an inert and/or conductive material depending on the application.

With reference to FIGS. 1, 2a, 2b, 3a, 3b, 4, 5a, 5b and 8, a first embodiment of the system for regenerating, or repairing, or replacing, tendon and/or ligamentous tissue of the present invention comprises:
the device of the present invention according to its second embodiment, namely, a tweezer (10)-screw (30) assembly as described above;
at least one filamentary structure comprising a plurality of nanofiber assemblies obtained by electrospinning, said plurality of assemblies being arranged to form a single bundle.

The pass bundle is wrapped to the capstan (12) of the tweezer itself (10). Additional nanofiber bundles, in addition, may pass through the pores of the porous portion (13, 13′) of the tweezer (10) and, possibly, also through the pores of the porous portion (33, 33′) of the screw (30), as well as through the hole (11) in the center of the capstan (12). With reference to FIGS. 6, 7a, 7b and 8, a second embodiment of the system for regenerating, or repairing, or replacing, tendon and/or ligament tissue of the present invention comprises:
the device of the present invention according to its sixth embodiment, namely, a plate (20) as described above;
at least one filamentary structure comprising a plurality of nanofiber groups obtained by electrospinning, said plurality of groups being arranged to form a single bundle.

The bundle is wrapped at each capstan (22, 22′, 22″) of the plate (20). Additional nanofiber bundles, in addition, can pass through the pores of the porous portion (24) of the plate (24), as well as through the holes (21, 21′, 21″) in the center of the capstans (22, 22′, 22″)

Finally, both the first and second embodiments of the system of the present invention can be used to simulate tendon and/or ligamentous tissue and thus be part of a prosthetic actuator or robotic system.

### Examples

Finite Element Simulation

In order to have a validation of the designed device and to study the gradient of deformability the tweezer-screw assembly, a finite element simulation was performed using Ansys Workbench 2019 R3 software. Geometric CAD models were imported from SolidWorks with some simplifications on the geometry. Due to the difficulties of the meshing operation, after verifying the condition of the screw as nearly unloaded, it was decided to neglect the trabecular model.

To load the tweezer-screw assembly as in an operational working condition, a tendon-inspired system was modeled as a simplified nylon bundle assembly. To simplify the model in this simulation, only the nylon side (the working part of the biphasic bundle attachment) was considered. The bundle assembly was modeled using a hyperelastic Nylon material. The geometry of the bundles was based on a cylinder having, at the end, a ring, which surrounds the tweezer inside the screw. The main section of the cylindrical bundle has a diameter of 3.80 mm before splitting into two half-bundles. In order to have a constant cross section for the entire bundle, the half-dashes were modeled as having an elliptical cross section with a minor and major semi-axis of 1.20 mm and 1.50 mm, respectively. The two half-dashes surround the tweezer and then join together in the main circular section.

In addition, a baseplate was introduced as a support for the assembly. PLA plastic (material present in the Ansys library, E=3.5 GPa and σY=54.1 MPa) was used as the material of the base and the tweezer in the simulation. Considering the absence of the trabecular model, reduced properties were assigned to the screw, to confer greater strain, with a Young's modulus of 2.4 GPa.

The baseplate was used as a fixed support to which the Voronoi porosity screw is attached, allowing, thus testing the thread resistance under load. A load was applied to the opposite side of the tweezer cross-section. To simplify the finite element simulation, a quarter of the CAD model was used. This simplification was done everywhere less than where the holes on the tweezer and the screw did not exhibit true symmetries. This assumption was imposed to significantly reduce the computational time.

The model was subjected to a “meshing” procedure using the Ansys Tetrahedra method. Tetrahedra with a maximum size of 0.40 mm were used for meshing the tweezer and the trabecular screw. To better study the trabecular surface subjected to higher loading, tetrahedral “meshes” with a maximum size of 0.25 mm were used for the tweezer. For the baseplate, tetrahedral “meshes” with a maximum dimension of 0.50 mm were used. The entire “mesh” of the model has 207402 nodes and 134632 elements, with a “meshing” quality of 0.82. Only linear elements were used.

Two different contacts were used to better simulate the interaction between the four parts:
friction with a coefficient of friction=0.3, between the base and the screw in the threaded connection area, and, between the screw and the tweezer, to simulate interference conditions;
friction with a coefficient of friction=0.4, between the tweezer and the bundle assembly, on the faces where wrapping occurred.

Contact between the trabecular screw and the tweezer was imposed in the area where the groove pitch provides axial clamping of the tweezer and between the inside of the screw and the top face of the tweezer. Lateral contact was imposed to prevent instability during compression of the tweezer arms. The model was clamped using a fixed support on the bottom face of the base. The load was applied as a force on the upper section of the bundle, simulating the force applied by the connected muscle-inspired bundle.

Simulation Results

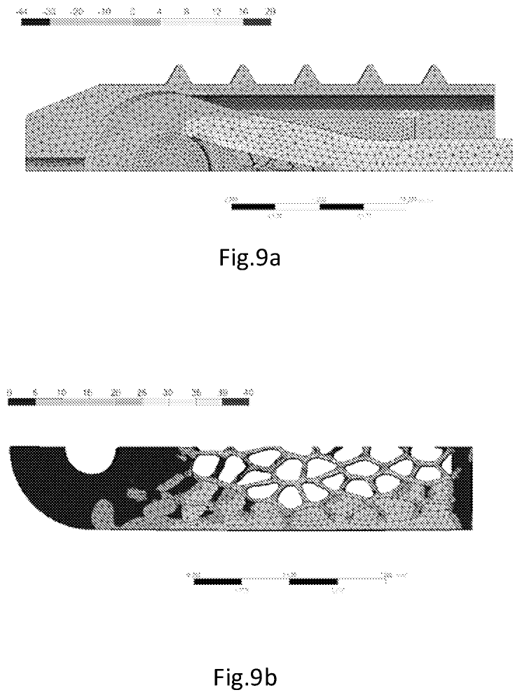

The finite element simulation was performed in 2 hours and 24 minutes. Due to the risk of elastic instability for a fully trabecular tweezer, a partial trabecular tweezer was used in this simulation. This tweezer exhibited compressive loading but no signs of impending instability. The normal stress results are shown in FIG. 9a.

As imagined, the normal stress presents the highest value at the point where there is detachment between the bundle assembly and the capstan, due to a concentration of stresses. A normal stress concentration factor of 3.24 can be calculated.

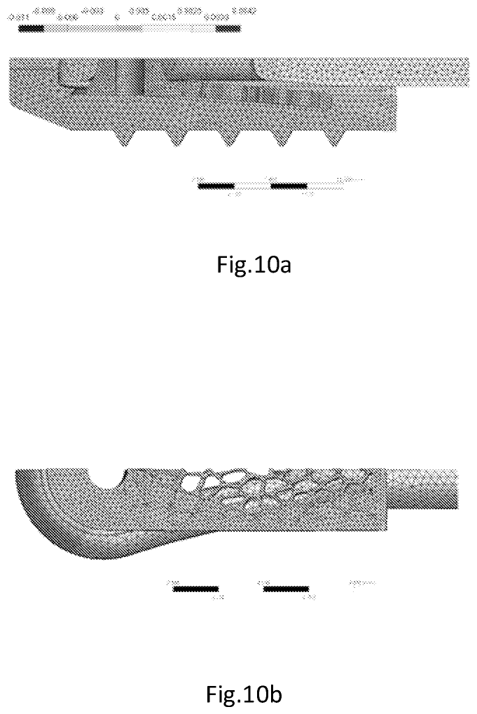

The normal strain analysis presented a high strain of the bundle assembly, while the trabecular screw proved to be almost non-deformable. The tweezer, on the other hand, presented an average strain compared to that of the other two components (screw and bundle assembly), thus providing for the desired strain gradient of the bioinspired junction. The normal strains of the tweezer and the bundle assembly are shown in more detail in FIGS. 10a and 10b.

1. A device for interfacing at least one filamentous structure, with real or simulated biological tissue, comprising at least one body for anchoring the filamentous structure, and said device comprising:
at least one capstan configured for wrapping of the filamentous structure; and
at least one porous portion having a trabecular structure and including at least a first porous zone having a porosity comprised between 1% and 98% and a pore size comprised between 0.1 μm and 800 μm and a second porous zone having a pore size comprised between 1 μm and 980 μm, wherein the difference between the first porous zone and the second porous zone being comprised between 1% and 98% and being such as to ensure a gradient of deformability at least between the first porous zone and the second porous zone.

2. A device for interfacing at least one filamentous structure, with real or simulated biological tissue, comprising:
a first body for anchoring the filamentous structure, wherein said first body comprises:
at least one capstan configured for wrapping of the filamentous structure, and
at least one porous portion having a trabecular structure; and
a second hollow body including at least one porous portion having a trabecular structure, wherein the first body for anchoring the filamentous structure is housed inside of the second hollow body;
wherein
the first body is conformed as a tweezer comprising a first flat arm and a second flat arm;
wherein the at least one porous portion of the first body includes a porosity comprised between 1% and 98% and a pore size comprised between 0.1 μm and 800 μm;
wherein the at least one porous portion of the second body includes a pore size comprised between 1 μm and 980 μm, and the difference of the porosity of at least one porous portion of the first body, and the porosity of the at least one porous portion of the second body being comprised between 1% and 98%, and being such as to ensure a gradient of deformability at least between the first body and the second body.

3. The device according to claim 1, wherein the structure of the at least one porous portion follows a Voronoi tessellation that is projected onto the surface of the body for anchoring the filamentous structure.

4. The device according to claim 1, wherein the body for the anchoring the filamentous structure is conformed as a tweezer comprising a first flat arm and a second flat arm, wherein said arms are joined to one another by means of the capstan.

5. The device according to claim 1, wherein the body has the shape of a plate provided with a plurality of capstans.

6. The device according to claim 2, wherein the at least one porous portion of the second body comprises a first porous zone and a second porous zone, wherein the pore size of said zones being comprised between 1 μm and 980 μm, and the difference between the porosity of the first porous zone and the porosity of the second porous zone being comprised between 1% and 98%.

7. The device according to claim 4, wherein said device includes a second hollow body including at least one porous portion having a trabecular structure, wherein the body for anchoring the filamentous structure is housed inside of the second body.

8. The device according to claim 7, wherein the body for anchoring the filamentous structure is embedded inside of the second body.

9. The device according to claim 7, wherein the second body has the shape of a threaded screw.

10. The device according to claim 7, wherein the at least one porous portion of the second body comprises a first porous zone and a second porous zone, and wherein the difference between the porosity of the first porous zone and the porosity of the second porous zone of the second body is between 1% and 98%.

11. The device according to claim 1, wherein at least one part of the device is made of at least one material selected from the group consisting of bioreabsorbable material, biocompatible material, inert material, and conductive material.

12. The device according to claim 11, wherein the at least one material is selected from the group consisting of: polyesters, polyurethanes, polyanhydrides, polycarbonates, polyamides, polyolefins, fluorinated polymers, polyester copolymers, polyurethane copolymers, polyanhydrides copolymers, polycarbonates copolymers, polyamide copolymers, polyolefin copolymers, fluorinated polymer copolymers, polysaccharides, proteins, polyesters, polypeptides, polysaccharide copolymers, protein copolymers, polyester copolymers, polypeptide copolymers, and metal and ceramic material.

13. A system for at least one of regeneration, repair, replacement, and simulation of tendon and/or ligament tissue, the system comprising:
a device for interfacing at least one filamentous structure, having with real or simulated biological tissue, comprising at least one body for anchoring the filamentous structure having at least one capstan and having at least one porous portion; and
at least one filamentous structure comprising a plurality of electrospun nanofiber groups;
wherein said plurality of electrospun nanofiber groups are arranged to form a single bundle; and
wherein said bundle is wrapped to the capstan.

14. The system according to claim 13, wherein the electrospun nanofiber bundle passes into the pores of the porous portion.

15. The system according to claim 14, wherein the system is configured to be used with at least one of a prosthetic actuator and a robotic system.
